(19) United States
(12) Patent Application Publication
(10) Pub. No.: US 2010/0076072 A1
(43) Pub. Date: Mar. 25, 2010

(54) COMPOSITIONS AND METHODS OF VITAMIN E ADMINISTRATION

(75) Inventor: Joan Cook-Mills, Aurora, IL (US)

Correspondence Address: Casimir Jones, S.C., 2275 DEMING WAY, SUITE 310, MIDDLETON, WI, 53562, US

(73) Assignee: NORTHWESTERN UNIVERSITY, Evanston, IL (US)

(21) Appl. No.: 12/510,078

(22) Filed: Jul. 27, 2009

Related U.S. Application Data
(60) Provisional application No. 61/083,780, filed on Jul. 25, 2008.

(51) Int. Cl. A61K 31/355; A61P 29/00; A61P 11/06

(52) U.S. Cl. 514/458

(57) ABSTRACT

The present invention relates to compositions and methods of vitamin E administration. In particular, the present invention provides opposing effects, relative to inflammation, from the administration of two major forms of vitamin E (α-tocopherol and γ-tocopherol).

The present invention claims priority to U.S. Provisional Patent Application Ser. No. 61/083,780, filed Jul. 25, 2008, the disclosure of which is herein incorporated by reference in its entirety.

### STATEMENT REGARDING FEDERALLY SPONSORED RESEARCH OR DEVELOPMENT

This invention was made with government support under National Institutes of Health Grant No. R01 HL069428. The government has certain rights in the invention.

### FIELD OF THE INVENTION

The present invention relates to compositions and methods of vitamin E, and related compound, administration. In particular, the present invention provides opposing effects, relative to inflammation, from the administration of two major forms of vitamin E (α-tocopherol and γ-tocopherol).

### BACKGROUND

Low vitamin E levels in patients correlate with asthma (Kalayci et al. 2000. Turk. J. Peds. 42: 17-21., Kelly et al. 1999. Lancet 354: 482-483., herein incorporated by reference in their entireties). Furthermore, after airway challenge, lung levels of vitamin E are reduced (Shvedova et al. 1995. Toxicol. Appl. Pharmacol. 132: 72-81., Schunemann et al. 2001. Am. J. Respir. Crit. Care Med. 163: 1246-1255., herein incorporated by reference in their entireties). Since, vitamin E levels are low in asthmatics and vitamin E can reduce inflammation in animal models, an increase in vitamin E may be necessary to promote optimal health in combination with other regimens to treat asthma. Vitamin E has been suggested to exert anti-inflammatory actions and has been shown to be effective in reducing inflammation in experimental asthma in mice (Zheng et al. 1999. American Journal of the Medical Sciences 318:49-54) and in asthmatics in studies in Finland and Italy. Vitamin E trials in the United States and the Netherlands have failed to show benefit in asthma. There are similar contradictory outcomes for vitamin E clinical trials of atherosclerosis.

### SUMMARY OF THE INVENTION

In some embodiments, the present invention provides a method of treating inflammation in a subject comprising providing a tocopherol composition wherein the tocopherol composition has greater than 90% α-tocopherol and less than 10% γ-tocopherol, and administering the tocopherol composition to a subject under conditions such that inflammation is reduced. In some embodiments, the tocopherol composition comprises greater than 95% α-tocopherol and less than 5% γ-tocopherol. In some embodiments, the tocopherol composition comprises greater than 99% α-tocopherol and less than 1% γ-tocopherol. In some embodiments, the tocopherol composition comprises greater than 99.9% α-tocopherol and less than 0.1% γ-tocopherol. In some embodiments, the tocopherol composition contains no detectable γ-tocopherol (e.g., as measured by standard analytical techniques). In some embodiments, the tocopherol composition comprises a delivery vehicle which contains additional components configured to deliver the tocopherol composition to a subject. In some embodiments, the delivery vehicle comprises an oral delivery vehicle. In some embodiments, the delivery vehicle comprises a food supplement. In some embodiments, the delivery vehicle comprises a food product. In some embodiments, the tocopherol delivery vehicle comprises a dietary supplement.

In some embodiments, the tocopherol delivery vehicle comprises a drug vehicle. In some embodiments, the delivery vehicle comprises a pharmaceutical composition. In some embodiments, the inflammation results from asthma. In some embodiments, the inflammation results from atherosclerosis.

In some embodiments, the present invention provides a composition comprising an orally administratable composition (e.g., pill, capsule, food product, dietary supplement, beverage, spray, mist, etc.) supplemented by the addition of the α-tocopherol isoform of vitamin E. In some embodiments, the vitamin E is substantially free of γ-tocopherol.

### DESCRIPTION OF FIGURES

The foregoing summary and detailed description may be better understood when read in conjunction with the accompanying drawings which are included by way of example and not by way of limitation.

FIG. 1 shows the chemical structures of natural d-α-tocopherol and d-γ-tocopherol.

FIG. 2 shows a schematic for tocopherol treatments during antigen activation of experimental asthma.

FIG. 3 shows a) body and b) lung weights on day 21 for mice treated with tocopherols.

FIG. 4 shows tocopherol levels in a) lung tissue and b) plasma on day 21 for mice treated with tocopherols. Column a) mice were administered vehicle. Column b) mice were administered α-tocopherol. Column c) mice were administered γ-tocopherol. Column d) mice were administered both α- and γ-tocopherol.

FIG. 5 shows the opposite effects of α- and γ-tocopherol on leukocyte infiltration into the bronchoalveolar lavage (BAL).

FIG. 6 shows the opposite effects of α- and γ-tocopherol on eosinophil infiltration into the lung tissue, with no effect of α- and γ-tocopherol on blood eosinophil numbers or OVA-specific serum IgE.

FIG. 7 shows the opposite effects of α- and γ-tocopherol on airway hyperresponsiveness.

FIG. 8 shows α- and γ-tocopherol treatments did not alter BAL cytokines, tissue eotaxins, endothelial cell VCAM-1 expression, or lung tissue PGE2.

FIG. 9 shows achievement of physiological tocopherol concentrations in mHEVa endothelial cells by treatment with α- or γ-tocopherol in vitro. a) α-tocopherol, b) γ-tocopherol treatment of mHEVa endothelial cells and c) α-tocopherol plus γ-tocopherol treatment of mHEVa endothelial cells in culture medium containing serum.

FIG. 10 shows the opposing effects of αT and γT on VCAM-1-dependent spleen cell migration in vitro; tocopherols directly regulate endothelial cells but not leukocytes during leukocyte migration. A-D)_Transwell assay for leukocyte migration with tocopherol left in the culture. Endothelial cells on transwells were incubated overnight with tocopherols and then spleen cells were added to the upper chamber of the transwells to examine cell migration. A) dose curve of αT. B) dose curve of γT treatment. C) dose curve of γT effects on αT (80 μM)-treated cells. D) endothelial cells were treated overnight with tocopherol and then in the presence or absence of blocking anti-VCAM-1 antibodies, spleen cell migration was determined. Anti-VCAM-1 antibodies were added every 4 hours as previously described (19). This figure further shows: x) Expression of VCAM-1 and MCP-1 by endothelial cells. x) Mean fluorescent intensity of anti-VCAM-1 or isotype antibody control labeled cells. x) MCP-1 in mHEVA culture supernatant. x-x) Tocopherol pretreatment of cells followed by washing before the start of the leukocyte migration assay with physiological laminar flow. x) Mice were treated with 2 mg tocopherol/day or vehicle (ethoxylated castor oil) for four days. Spleens were collected, red blood cells were lysed, and leukocyte tocopherol was determined. x) Spleen leukocytes from untreated mice or spleen leukocytes from mice treated with tocopherols as in panel x were added to the endothelial cells and examined for transendothelial migration at 15 minutes under physiological laminar flow. x) Spleen leukocytes from untreated mice or spleen leukocytes from mice treated with tocopherols as in panel x were added to the endothelial cells and examined for association with the endothelial cells at 2 minutes under physiological laminar flow. x,x) 85% confluent monolayers of mHEVa cells on slides were treated overnight with the tocopherols at the concentrations indicated to achieve physiological concentrations of tocopherols (FIGS. 5A-C) and then the endothelial cells were washed before addition of spleen cells. For spleen cells, spleen cells were isolated from untreated mice, the spleen red blood cells were lysed, and these spleen leukocytes were added to the endothelial cells to examine leukocyte transendothelial migration under physiological laminar flow. x) Leukocyte transendothelial migration at 15 minutes under laminar flow. x) Leukocyte association with endothelial cells at 2 minutes under laminar flow. n=3-5. *, p<0.05 compared to DMSO vehicle-treated controls. DMSO (0.02%) did not affect migration (data not shown).

FIG. 11 shows that tocopherols regulate VCAM-1 activation of endothelial cell PKCα. Endothelial cells were pretreated overnight with physiological concentrations of tocopherols (80 μM α-tocopherol and/or 2 μM γ-tocopherol). Endothelial cell VCAM-1 was stimulated with anti-VCAM-1 and a secondary antibody for 10 minutes. Phosphorylation of PKCα-Thr⁶³⁸ was examined by western blot, developed using an ECL kit, and analyzed by densitometry as in the methods section. These concentrations of α-tocopherol (80 μM) and γ-tocopherol (2 μM) were optimal for their inhibition or enhancement, respectively, of VCAM-1 activation of PKCα (data not shown). Shown are representative blots and data from 5 experiments. *, p<0.05 compared to nonstimulated groups. **, p<0.05 compared to nonstimulated groups and p<0.07 compared to DMSO-treated/anti-VCAM-stimulated group.

FIG. 12 shows α- and γ-tocopherol did not alter endothelial cell a) VCAM-1 expression or b) MCP-expression.

FIG. 13 shows a) tocopherol content in cooking oils and b) human plasma γ-tocopherol levels in several countries.

FIG. 14 shows sample ratios of α-tocopherol to γ-tocopherol for several common types of nutritional supplements represented in International Units (IU) and percent daily value.

### DETAILED DESCRIPTION

The present invention relates to compositions and methods of vitamin E, and related compounds and administration. In particular, the present invention provides opposing effects, relative to inflammation, from the administration of two major forms of vitamin E (α-tocopherol and γ-tocopherol) (Berdnikovs et al. The Journal of Immunology, 2009, 182: 4395-4405., herein incorporated by reference in its entirety). In some embodiments, the present invention provides anti-inflammatory benefits upon administration of D-α-tocopherol to a subject (e.g. human subject). In some embodiments, the presence of isoform D-γ-tocopherol, or sufficiently high levels of D-γ-tocopherol, elevates inflammation in experimental asthma. In some embodiments, D-γ-tocopherol, at as little as 10% the concentration of D-α-tocopherol, ablates the anti-inflammatory benefit of the D-α-tocopherol isoform. In some embodiments, the present invention provides compositions and methods for the preparation and administration of drugs, foods, supplements, and other compositions containing excess α-tocopherol over γ-tocopherol, excess α-tocopherol, reduced γ-tocopherol, α-tocopherol in place of γ-tocopherol, only α-tocopherol and free or substantially free of γ-tocopherol, etc. In some embodiments, experiments performed during development of embodiments of the present invention demonstrate the health benefits (e.g. anti-inflammatory benefits) of α-tocopherol-rich compositions lacking or substantially lacking γ-tocopherol.

Low vitamin E levels in patients correlate with asthma. Furthermore, after airway challenge, lung levels of vitamin E are reduced. Since vitamin E levels are low in asthmatics and since vitamin E can reduce inflammation in animal models, it is contemplated that an increase in vitamin E may be necessary to promote optimal health in combination with other regimens to treat asthma. It has been contemplated that vitamin E exerts anti-inflammatory actions, and it has been shown to be effective in reducing inflammation in experimental asthma in mice and in asthmatics in studies in some countries in Europe (Finland and Italy). Vitamin E trials in the United States and the Netherlands have failed to show benefit in asthma. There are similar contradictory outcomes for vitamin E in clinical trials of atherosclerosis. These clinical and animal studies have focused on one form of vitamin E, α-tocopherol, without analysis of other forms of vitamin E in the diet. The two major forms of vitamin E in the diet are α-tocopherol and γ-tocopherol. γ-tocopherol is the major form of vitamin E in the diet of Americans but is not in abundance in most diets of Europeans. Several reports indicate that plasma levels of γ-tocopherol in Americans and in Dutch is 2-5 times higher than most Europeans. Although γ-tocopherol is the major form in most American diets, retention of γ-tocopherol in tissues is 10% that of α-tocopherol. This is contemplated to be a result of preferential transfer of α-tocopherol in the liver by α-tocopherol transfer protein.

A consistent feature of asthma is the infiltration of eosinophils. The extent of eosinophilia varies among asthmatic individuals as does the severity of the increased airway response to inhaled antigens. Other components of asthma include infiltration of neutrophils, monocytes, and lymphocytes and signals that cause airway hyperresponsiveness (AHR). The mechanisms underlying leukocyte recruitment to the lung following allergen exposure are complex, involving the coordinate actions of adhesion molecules, chemokines, and cytokines. During an allergen challenge in asthma, tissue cells produce cytokines such as IL-1 and IL4 that activate the endothelium to express adhesion molecules. The adhesion molecules mediate binding of leukocytes in the blood. The adhesion molecules on the endothelial cells and the corresponding receptors on the leukocytes stimulate intracellular signals in the leukocytes and the endothelial cells. These signals are required for leukocyte migration and localized endothelial cell retraction for the opening of a passageway. The leukocytes bound to the endothelium migrate through this passageway in the endothelium and migrate into the tissue in response to a chemokine gradient. Then, the resident tissue cells, infiltrating leukocytes, and cells in the draining lymph nodes produce cytokines that establish microenvironments for the regulation of the immune response in the tissues. An experimental mouse model of asthma is intraperitoneal (i. p.), ovalbumin (OVA) sensitization followed by OVA challenge via inhalation. Eosinophil recruitment to the lung in murine models of allergic inflammation is dependent upon eosinophil binding to the adhesion molecule vascular cell adhesion molecule-1 (VCAM-1) on endothelial cells. The infiltration of other leukocytes in experimental asthma is mediated by binding to ICAM-1 or PECAM-1.

Experiments conducted during the development of embodiments of the invention demonstrated that VCAM-1 stimulates signals through endothelial cell Nox2 generation of reactive oxygen species in vitro. Furthermore, endothelial cell gp91 phox (the catalytic subunit of Nox2) is required for VCAM-1-dependent extravasation of eosinophils and airway hyperresponsiveness in experimental asthma using chimeric gp91 phox −/− mice. Experiments conducted during the development of embodiments of the invention have also demonstrated that the antioxidant bilirubin inhibits VCAM-1 dependent signaling in endothelial cells and inhibits eosinophilia in experimental asthma. Although bilirubin inhibits inflammation in experimental asthma, administration of bilirubin to patients is not ideal. Since experiments conducted during the development of embodiments of the invention demonstrated that VCAM-1-stimulated endothelial cell Nox2 generation of reactive oxygen species is required for eosinophil recruitment in experimental asthma, it was contemplated that the antioxidant vitamins α-tocopherol or γ-tocopherol could modulate AHR and VCAM-1-dependent infiltration of eosinophils into the lungs of mice with experimental asthma.

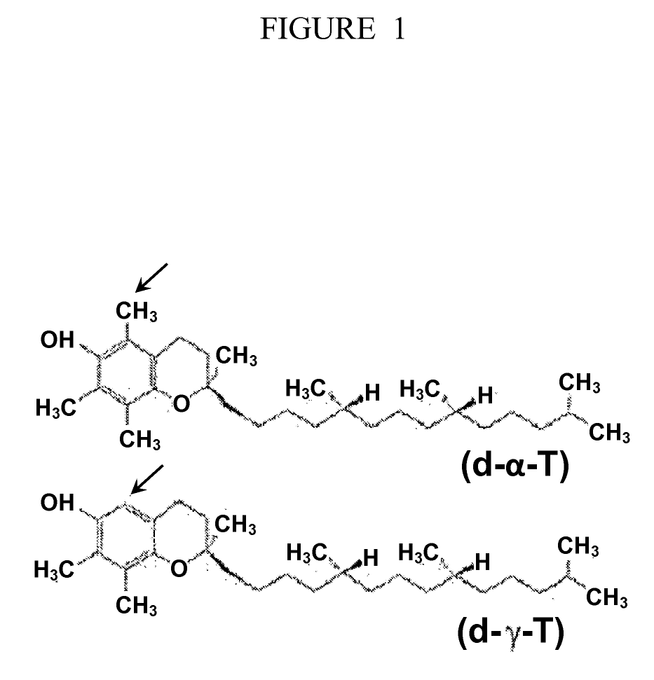

Vitamin E consists of eight isomers, four tocopherols (α-, β-, γ-, δ-tocopherol) and four tocotrienols (α-, β-, γ-, δ-tocotrienol). There are four natural tocopherols, d-α-tocopherol, d-β-tocopherol, d-tocopherol, and d-δ-tocopherol. In addition, there are synthetic forms of tocopherols. Synthetic α-tocopherol (d-α-tocopherol) contains 8 racemic isoforms, of which only one of these is the natural form of tocopherol. Synthetic d-γ-tocopherol also contains 8 racemic forms. These racemic synthetic tocopherols can also be synthesized as tocopherol acetates. Tocopherols are metabolized to tocopherol quinones and to CEHC (2,5,7,8,-tetramethyl-2(2′-carboxyethyl)-6 hydroxychroman) (Traber and Kayden. 1989. American Journal of clinical Nutrition 49:517-526, herein incorporated by reference in its entirety). Of these forms of tocopherol, experiments conducted during the development of the embodiments of present invention focused on two natural forms of vitamin E, d-α-tocopherol and d-γ-tocopherol, which differ by one methyl group (SEE FIG. 1). These two forms are the major forms consumed by Americans and are the most abundant forms in the tissues. d-γ-tocopherol is the most abundant tocopherol in the American diet as it is abundant in soy and vegetable (often soy) oil (Wolf. 2006. Nutr. Rev. 64: 295-299., herein incorporated by reference in its entirety). α-tocopherol is the predominant form in plasma and supplements and it has been extensively studied as an antioxidant to protect lipids from oxidation. α-tocopherol and γ-tocopherol have both antioxidant and non-antioxidant functions (Jiang et al. 2001. Am. J. Clin. Nutr. 74: 714-722., Azzi & Stocker. 2000. Prog. Lipid Res. 39: 231-255., herein incorporated by reference in their entireties).

Both γ-tocopherol and α-tocopherol are required for optimal health. Dietary tocopherols are taken up and transported through the lymph to blood and then to the liver where they are transfered to lipoproteins that can be taken up by tissues. Dietary tocopherols are taken up from the intestine by scavenger receptor class B type I on enterocytes in mice and about half of the uptake is by diffusion (Reboul et al. 2006. J Biol Chem 281:4739-4745., herein incorporated by reference in its entirety). Typical American diets are more abundant in γ-tocopherol than α-tocopherol. However, α-tocopherol is preferentially transferred to plasma lipoproteins by the liver α-tocopherol transfer protein (αTTP) (Wolf. 2006. Nutr. Rev. 64: 295-299., Leonard et al. 2002. Am J din Nutr 75:555-560., herein incorporated by reference in their entireties). αTTP transfers α-tocopherol to low density lipoprotein particles (LDL) and high density lipoprotein particles (HDL) that then enter the blood. It has been contemplated that αTTP can also carry significant quantities of γ-tocopherol into circulation because αTTP −/− mice have reduced γ-tocopherol that is not recovered by an elevated γ-tocopherol diet (Leonard et al. 2002. Am J din Nutr 75:555-560., herein incorporated by reference in its entirety). Tocopherols can be transferred from LDL or HDL to endothelial cells by plasma phospholipid transfer protein. In addition, tocopherols can be taken up by cells through their scavenger receptors or the lipoprotein lipase pathway. After uptake, the tocopherols are found in cell membranes as they are lipid vitamins. Different diet levels of α-tocopherol result in different tissue levels of α-tocopherol. Experiments conducted during the development of embodiments of the present invention demonstrated that γ-tocopherol elevates inflammation in experimental asthma. γ-tocopherol, at 10% the tissue concentration of α-tocopherol, opposes α-tocopherol inhibition of inflammation and airway hyperresponsiveness in experimental asthma. Furthermore, these tocopherols have direct effects on endothelial cell function during leukocyte recruitment.

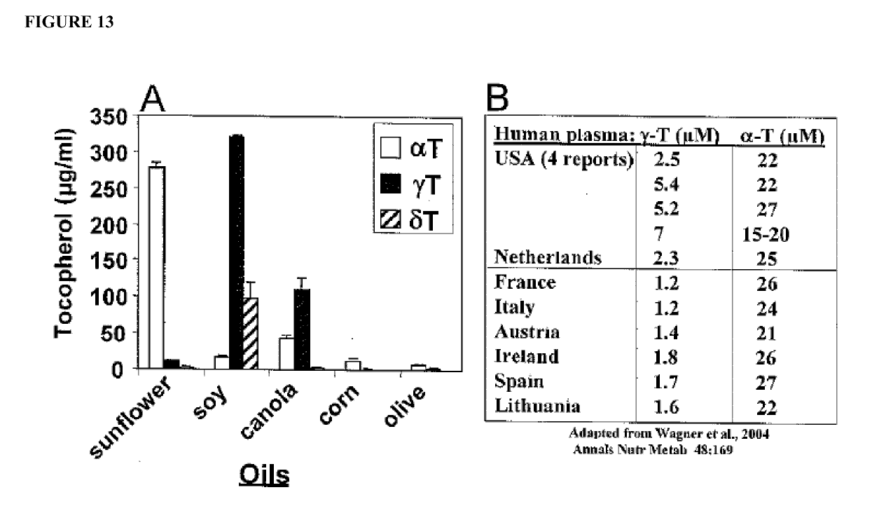

Experiments conducted during the development of embodiments of the invention demonstrated that the d-α-tocopherol form of vitamin E inhibits leukocyte infiltration into the lung, and reduces airway hyperresponsiveness (AHR) in experimental asthma. In contrast, these responses are exacerbated by the d-γ-tocopherol form of vitamin E. D-γ-tocopherol, at 10% the tissue concentration of d-α-tocopherol, blocks the d-α-tocopherol inhibition of leukocyte infiltration into the lung. Pre-treatment of endothelial cells in vitro with d-α-tocopherol or d-γ-tocopherol inhibits or elevates, respectively, leukocyte transendothelial migration. in vitro pretreatment with d-γ-tocopherol blocks the d-α-tocopherol inhibition of leukocyte migration. Both of these tocopherols modulate leukocyte migration by directly affecting the endothelial cells. Natural d-α-tocopherol and natural d-γ-tocopherol differ in structure by only one methyl group, but have opposing effects on inflammation. Experiments were conducted during the development of embodiments of the invention to determine the levels of α-tocopherol and γ-tocopherol in dietary oils (FIG. 13A). The American diet is rich in γ-tocopherol found in soy oil, the major form of vegetable oil in the United States. In contrast, γ-tocopherol is low in other oils commonly used in European countries (SEE FIG. 13A). Experiments conducted during the development of embodiments of the invention on α-plus γ-tocopherol treatment of mice demonstrated that there was little benefit of α-tocopherol for asthmatics in the presence of elevated plasma γ-tocopherol. Therefore, it is contemplated that differences in outcome of clinical reports on vitamin E modulation of asthma in European countries and the Unites States may reflect the opposing effects of α- and γ-tocopherol forms of vitamin E consumed in diets and supplements. The opposing effects of tocopherols observed in experiments conducted during the development of embodiments of the invention have implications on the interpretation of clinical studies with mixed tocopherols in supplements and diets. Low α-tocopherol levels in patients correlate with asthma and airway challenge lowers lung levels of α-tocopherol in humans and in guinea pigs. Therefore, it is contemplated that since α-tocopherol levels are low in asthmatics and since α-tocopherol can reduce inflammation, an increase in α-tocopherol in the presence of low γ-tocopherol may be necessary to promote optimal health in combination with other regimens to treat inflammatory disease. Experiments conducted during the development of embodiments of the invention on tocopherol modulation of inflammation suggest that it is important to examine the forms of tocopherol used in animal studies.

Experiments conducted during the development of embodiments of the invention demonstrate that, consistent with a benefit of α-tocopherol during inflammation, a deficiency in α-tocopherol elevates inflammation. Mice deficient in liver α-tocopherol transfer protein (αTTP) had reduced tissue α-tocopherol and elevated inflammation in experimental asthma. In other animal models of inflammatory disease, administration of α-tocopherol also reduces inflammation. α-tocopherol reduces endotoxin-stimulated neutrophil infiltration into the lung. α-tocopherol reduces delayed-type-hypersensitivity responses (Ikarashi et al. 1998. Journal of Nutritional Science and Vitaminology 44:225-236., herein incorporated by reference in its entirety) and reduces high oxidation by ozone and smoke in the lung (Grievink et al. 1999. American Journal of Epidimiology 149:306-314., Kari et al. American Journal of Respitory Cell & Molecular Biology 17:740-747., Trenga et al. 2001. archives of Environmental Health 56:242-249., Romieu et al. 2002. American Journal of Respiratory and Critical care Medicine 166:703-709., herein incorporated by reference in their entireties). In atherosclerosis models, α-tocopherol has been reported to inhibit adhesion molecule expression. α-tocopherol consistently reduces inflammation in mouse models in the absence of consumption of γ-tocopherol as mouse chow contains low to no γ-tocopherol. Experiments conducted during the development of embodiments of the present invention demonstrate that, in mice on chow diet, administration of purified d-a-tocopherol blocks leukocyte infiltration and AHR whereas purified d-γ-tocopherol elevates these responses in experimental asthma.

Experiments conducted during the development of embodiments of the present invention have identified novel opposing functions for the major forms of vitamin E consumed by Americans. These opposing effects are consistent with the differential effects of vitamin E in studies of Americans versus Europeans and in studies of Americans versus animals. Experiments conducted during the development of embodiments of the present invention demonstrated a mechanism for α-tocopherol and γ-tocopherol modulation of inflammation by a direct effect of the tocpoherols on the endothelium, although the present invention is not limited to any particular mechanism of action and an understanding of the mechanism of action is not necessary to practice the present invention. The endothelium has an active role in leukocyte migration since adhesion molecules and endothelial cells activate the endothelium to open a passageway through which the leukocytes can migrate. The Experiments conducted during the development of embodiments of the present invention demonstrating opposite functions of α-tocopherol and γ-tocopherol in vivo have important implications an studies that analyze effects of mixed tocopherols on disease (Dietrich et al. 2006. J. Am. Coll. Nutr. 25: 292-299., Dietrich et al. 2002. Cancer Epidemiol Biomarkers Prey 11:7-13., herein incorporated by reference in their entireties).

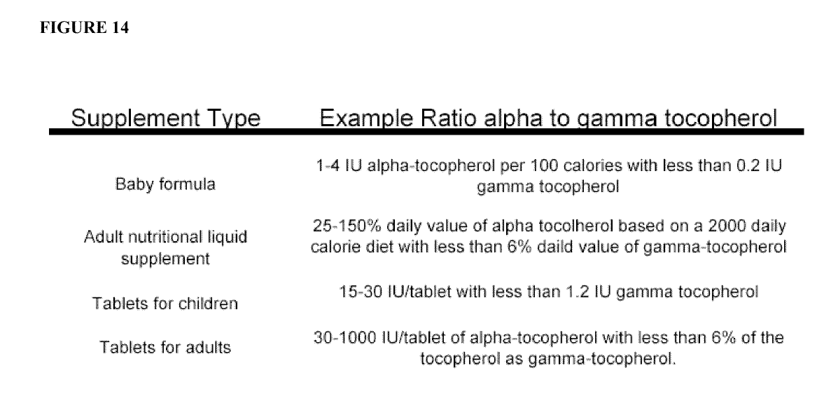

In some embodiments, the present invention provides methods for preparing food products (e.g., solid foods, beverages, etc.) and/or nutritional supplements that contain α-tocopherol, but are relatively low in γ-tocopherol (e.g., less than 10%, . . . , 5%, 4%, 3%, 2%, 1%, 0.5%, 0.2%, 0.1%, 0.05%, . . . undetected % γ-tocopherol relative to α-tocopherol). In some embodiments, the present invention provides compositions (e.g. food products (e.g., solid foods, beverages, etc.), nutritional supplements, etc.) that contain α-tocopherol, but are low in γ-tocopherol (e.g., less than 10%, . . . , 5%, 4%, 3%, 2%, 1%, 0.5%, 0.2%, 0.1%, 0.05%, . . . undetected % γ-tocopherol relative to α-tocopherol). FIG. 14 shows several non-limiting examples of products containing a high ratio of α-tocopherol to γ-tocopherol. In some embodiments, a food product that otherwise contains an oil rich in γ-tocopherol (e.g., soy and vegetable oils) is treated or manufactured to reduce or eliminate the γ-tocopherol in the food product. For example, in some embodiments, food products that would normally contain oil rich in γ-tocopherol are prepare with a substitute oil low in or free of γ-tocopherol. Some natural oils, such as sunflower oil, have a lower ratio of γ-tocopherol to a tocopherol than other oils (e.g., soy or canola). However, synthetic oils may be used that are free of or essentially free of γ-tocopherol.

In some embodiments, a food product naturally high in γ-tocopherol is super-supplemented with high levels of α-tocopherol so as to increase the ratio of α-tocopherol to γ-tocopherol (e.g., to greater than 1:1, 2:1, 5:1, 10:1, 20:1, 50:1, etc.). For example, a food product containing an oil with a 1:4 ratio with a total of 5 mg of vitamin E (i.e., 4 mg γ-tocopherol and 1 mg a tocopherol) may be supplemented with 100 mg of essentially pure α-tocopherol to produce a product that has a ratio of approximately 25:1. In some embodiments, a food product containing an oil with a high ratio of γ-tocopherol to α-tocopherol (e.g., canola oil or soy oil), but lacking an oil with high ratio of α-tocopherol to γ-tocopherol is supplemented with an oil with high ratio of α-tocopherol to γ-tocopherol (e.g., sunflower oil). For example, a product containing soy or canola oil, but normally lacking sunflower oil, may be manufactured with a lower percentage of soy or canola oil and addition of sunflower oil.

In some embodiments, a patient suffering from an inflammatory condition or susceptible to an inflammatory condition (e.g., a respiratory inflammatory condition), such as asthma, are provided a diet high in vitamin E, but low in or free of γ-tocopherol. In some embodiments, the patient if provided with a manufactured food product enriched with α-tocopherol at 15 mg or higher (e.g., 50 mg, 100 mg, 200 mg, 400 mg, 600 mg, 1000 mg, etc.) having low percentage of γ-tocopherol (e.g., less than 10%, . . . , 5%, 4%, 3%, 2%, 1%, 0.5%, 0.2%, 0.1%, 0.05%, . . . undetected %). In some embodiment some γ-tocopherol is present, but the amount is low (e.g., greater than 0.1% but lower than 10%, greater than 0.5% but lower than 5%, greater than 1% but lower than 5%, etc.). In some embodiments, the patient is provided with a daily diet with α-tocopherol at 15 mg or higher having low percentage of γ-tocopherol.

In some embodiments, vitamin E low having low percentage of γ-tocopherol is provided to the patient via injection, IV, or other non-oral means.

Experiments performed during development of embodiments of the present invention indicate that γ-tocopherol has an exacerbatory effect on arthritis.

In some embodiments, the level of γ-tocopherol in a patient (e.g., asthma patient) is detected or monitored to determine whether alterations in diet or treatment are needed to manage a disease. In some embodiments, levels (e.g., blood, plasma, or tissue levels) are monitored prior to any diet modification. In some embodiments, levels are monitored following diet modification. In some embodiments, if levels are considered too high, further diet or treatment modification is made. In some embodiments, absolute levels of γ-tocopherol are measured. In some embodiments ratios of γ-tocopherol to α-tocopherol are determined.

In some embodiments, a subject is provided with vitamin E low in γ-tocopherol prior to being exposed to an environment or procedure (e.g., medical procedure) likely to provoke an inflammatory response (e.g., a respiratory inflammatory response, e.g., asthma). Such exposures include, but are not limited to, allergens (e.g., household pests, grass pollen, pet epithelial cells), air pollution, volatile organic compounds, medications (e.g., aspirin, beta blockers, penicillin, etc.), foods (e.g., milk, peanuts, eggs), fossil fuels, sulfites, chloramines, viral respiratory infections, exercise, hormonal changes, emotional stress, and cold weather.

As used herein, the term “food product” refers to any food or feed suitable for consumption by humans, non-ruminant animals, or ruminant animals. The “food product” may be a prepared and packaged food (e.g., mayonnaise, salad dressing, spread, peanut butter, bread, cereals, shake, or cheese food) or an animal feed (e.g., extruded and pelleted animal feed or coarse mixed feed). “Prepared food product” means any pre-packaged food approved for human consumption.

As used herein, the term “foodstuff” refers to any substance fit for human or animal consumption.

In some embodiments, the present invention provides pharmaceutical and/or supplement compositions. The pharmaceutical and/or supplement compositions of the present invention can be administered in a number of ways depending upon whether local or systemic treatment is desired and upon the area to be treated. Administration can be topical (including ophthalmic and to mucous membranes including vaginal and rectal delivery), pulmonary (e.g., by inhalation or insufflation of powders or aerosols, including by nebulizer; intratracheal, intranasal, epidermal and transdermal), oral or parenteral. Parenteral administration includes intravenous, intraarterial, subcutaneous, intraperitoneal or intramuscular injection or infusion; or intracranial, e.g., intrathecal or intraventricular, administration. Compositions and formulations for topical administration can include transdermal patches, ointments, lotions, creams, gels, drops, suppositories, sprays, liquids and powders. Conventional carriers; aqueous, powder, or oily bases; thickeners; and the like can be necessary or desirable. Compositions and formulations for oral administration include powders or granules, suspensions or solutions in water or non aqueous media, capsules, sachets or tablets. Thickeners, flavoring agents, diluents, emulsifiers, dispersing aids or binders can be desirable. Compositions and formulations for parenteral, intrathecal or intraventricular administration can include sterile aqueous solutions that can also contain buffers, diluents and other suitable additives such as, but not limited to, penetration enhancers, carrier compounds and other pharmaceutically acceptable carriers or excipients. Pharmaceutical and/or supplement compositions of the present invention include, but are not limited to, solutions, emulsions, and liposome containing formulations. These compositions can be generated from a variety of components that include, but are not limited to, preformed liquids, self emulsifying solids and self emulsifying semisolids.

The pharmaceutical and/or supplement formulations of the present invention, which can conveniently be presented in unit dosage form, can be prepared according to conventional techniques well known in the pharmaceutical/nutriceutical industries. Such techniques include the step of bringing into association the active ingredients (e.g. α-tocopherol) with the pharmaceutical carrier(s) or excipient(s). In general the formulations are prepared by uniformly and intimately bringing into association the active ingredients (e.g. α-tocopherol) with liquid carriers or finely divided solid carriers or both, and then, if necessary, shaping the product. The compositions of the present invention can be formulated into any of many possible dosage forms such as, but not limited to, tablets, capsules, liquid syrups, soft gels, suppositories, and enemas. The compositions of the present invention can also be formulated as suspensions in aqueous, non aqueous, oil-based, or mixed media. Suspensions can further contain substances that increase the viscosity of the suspension including, for example, sodium carboxymethylcellulose, sorbitol and/or dextran. The suspension can also contain stabilizers. In one embodiment of the present invention the pharmaceutical compositions can be formulated and used as foams. Pharmaceutical foams include formulations such as, but not limited to, emulsions, microemulsions, creams, jellies and liposomes. While basically similar in nature these formulations vary in the components and the consistency of the final product.

In some embodiments, formulations, carriers, and any additional ingredients added to the active ingredients (e.g. α-tocopherol) are low in γ-tocopherol, γ-tocopherol-free, or substantially free of γ-tocopherol.

### EXPERIMENTAL

### Example 1

### Compositons and Methods for Vitimin E Administration

Animals. BALB/c female mice (Jackson Laboratories, Bar Harbor, Me.) were used for OVA sensitization at 5 weeks old. BALB/c male mice (Jackson Labs) were a source of spleen cells used for the in vitro transendothelial migration studies.

Cells. The endothelial cell line mHEVa was derived from male BALB/c mouse axillary lymph nodes and cultured as previously described (Cook-Mills et al, 1996). The mHEVa cells were independently spontaneously immortalized but are not transformed. These mHEVa cells constitutively express VCAM-1. Single cell suspensions of spleen cells were prepared from freshly isolated male BALB/c mouse spleens as previously described. Where indicated, red blood cells were lysed by hypotonic shock (Cook-Mills et ai, 1996).

Reagents. Ethoxylated castor oil was composed of polyethoxylated castor oil (PEG35), 20% ethanol, and 1% benzyl alcohol. Vital E-300 I.U. was α-tocopherol in ethoxylated castor oil and we confirmed that Vital E-300 did not contain detectable γ-tocopherol or δ-tocopherol as we determined by hexane extraction and HPLC analysis as described below. γ-tocopherol (Sigma) was >99% pure. The purified tocopherols were diluted in ethoxylated castor oil to indicated concentrations for the in vivo studies. The purified tocopherols were diluted in DMSO (0.2%) for the in vitro studies. OVA administration. BALB/c female mice were maintained on chow diet. The mice were sensitized by intraperitoneal injection (200 μl) of OVA grade V (10 μg)/alum or saline/alum on days 0 and 7 (SEE FIG. 1). On days 13-20, these mice received daily subcutaneous injections (50 μl) of 2 μg natural tocopherol/ethoxylated castor oil or ethoxylated castor oil alone. This daily 2 μg of natural tocopherol is equivalent to the amount of tocopherol that mice consume from diets (4 g diet consumed/day×500 mg α-tocopherol/kg diet) that are commonly used to elevate tissue tocopherols in mice. The mice recieved 2 μg d-α-tocopherol, 2 μg d-γ-tocopherol, 2 μg d-α-tocopherol plus 2 μg d-γ-tocopherol or the vehicle exthoxylated castor oil. Ethoxylated castor oil is used in pharmaceutical formulations for viscous lipids and does not have side effects. Elevated tocopherol does not alter body weight or diet consumption. During the OVA responses in mice, tocopherol radicals are recycled by endogenously synthesized ascorbic acid. On days 16, 18, and 20, the mice were challenged with intranasal OVA fraction VI (150 μg) in saline or saline alone. On day 21, the mice were analyzed for airway responsiveness followed by collection of tissues.

Airway Hyperresponsiveness. Lung responsiveness was determined using methacholine as previously described. On day 21, the mice were analyzed for airway responsiveness by whole body plethysmology. The whole-body plethysmography on conscious, unrestrained mice was performed under continuous airflow conditions (Buxco, Troy, N.Y.). Continuous airflow was used to avoid the influence of conditioning on airway functions, causing aberrant measurements. Enhanced pause (Penh) values were calculated from the flow-derived whole-body plethysmography measurements done with continuous air flow. Penh is a unitless indicator of changes in airway resistance and correlates well with specific airway resistance. Baseline readings were averaged for 5 minutes. Saline or methacholine was nebulized into the airflow for 2-minute periods and the average Penh value calculated for this and the subsequent 8 minutes.

Tissue collection. After airway function analysis, mice were weighed, sacrificed and the lungs flushed twice with 0.8 ml PBS. The cells in this bronchoalveolar lavage (BAL) were cytospun onto slides, DiffQuik (Baxter) stained, and differential cell counts were performed. BAL supernatants were snap frozen and stored at −80° C. for cytokine analysis. Lungs were perfused free of blood and one quarter of the lung was tied off, collected, weighed, snap frozen, and stored at −80° C. for tocopherol analysis and PGE2 analysis. One quarter of the lung was collected and placed in RNA later for analysis of mRNA for eotaxin expression. The other half of the lung was injected with 40% freezing compound, collected and stored at −80° C. for tissue sections and histological staining Frozen sections were prepared rather than paraffin sections since the anti-mouse VCAM-1 antibodies only bind to VCAM-1 in frozen sections.

Tocopherol measurement. The α-tocopherol concentrations in lungs and plasma were measured by HPLC with an electrochemical detector. Lungs that are perfused blood-free were weighed and homogenized in absolute ethanol, 5% ascorbic acid on ice. The internal standard tocol is added to each lung to determine recovery. The homogenate or plasma was extracted with an equal volume of hexane with 0.37 wt % butylated hydroxytoluene to prevent oxidation and increase recovery of tocopherol. The samples were vortexed and then centrifuged for 10 minutes at 9,000×g at 4° C. The hexane layer was dried under nitrogen and stored at −20° C. and reconstituted in methanol. The samples were reconstituted in methanol and then tocopherols were separated by reverse phase C₁₈ HPLC column with 99% methanol-1% water as a mobile phase with detection with an electrochemical detector (potential 0.7V). A calibration curve was generated using purified tocopherol standards.

Cytokines and Chemokines The BAL supernatants were tested for levels of IL-4, IL-5, IFNγ and IL-2 using the Th1/Th2 CBA kit (BD Siosciences). Eotaxins were determined by quatitative PCR from lung tissue. Total RNA was isolated from 50-100 mg lung tissue using the QIAGEN Rneasy Fibrous Tissue Mini Kit. cDNA was prepared using a SuperScript II RNase H-Reverse Transcriptase kit (Invitrogen Corp.), and analyzed by PCR on an ABI 7300 Thermal Cycler (applied Biosystems). Taqman probes and Taqman Universal Master Mix were used as directed (Applied Biosystems) for eotaxin 1 and 2.

Expression of VCAM-1. VCAM-1 expression in frozen lung tissue was examined by immunofluorescence labeling and confocal microscopy as previously described. The fluorescence of anti-VCAM-1-labeled cells was quantified by calculating the sum of the endothelial cell-associated fluorescence pixel intensities/100>μm² for the vessel and lumen minus the sum for the lumen.

Tissue eosinophils. Eosinophils in lung tissues was examined by immunolabeling eosinophil granules with anti-major basic protein antibodies. The number of perivascular and peribronchial eosinophils was determined and presented as number of cells per high powered field OVA-specific IgE. OVA-specific IgE was determined by ELISA as previously described.

PGE2. The ethanol fraction from the lung extracts for tocopherol measurements was collected, dried under nitrogen and suspended in the buffer of the High Sensitivity PGE2 ELISA kit buffer (Cambridge Biosciences).

In vitro Transwell migration assay. mHEVa cells were grown to confluence on Transwells with 12 μm pores (Costar Cambridge, Mass.). The mHEVc cell monolayers block nonspecific accumulation of red blood cells in the lower transwell chamber and block FITC-albumin diffusion. Spleen cell migration is stimulated by mHEV cell secretion of the chemokine MCP-1. The number of lymphocytes that migrate is linear from 0-24 hrs followed by a plateau. Spleen cells that migrate are greater than 95% lymphocytes and the percentage of cells that migrate is consistent with other in vitro models with endothelial cell lines or cytokine-activated microvascular endothelial cells.

In vitro cell association and migration assays with laminar flow. The parallel plate flow chamber was used to examine migration under conditions of laminar flow. Endothelial cells were grown to confluence on slides and then the slide was placed in a parallel plate flow chamber. In vivo, in the absence of inflammation, the rapid fluid dynamics of the blood result in blood cells located midstream of the vascular flow. However, during inflammation, there is a change of fluid dynamics. With inflammation, vascular permeability increases yielding fluid flow from the blood into the tissues which contributes to contact of blood cells with the endothelium (“margination. There is also cell contact as the blood cells leave the capillaries and enter the postcapillary venules.

Therefore, spleen cells (3×10⁶) were added to the flow chamber (35 cm²) at 2 dynes/cm². The conditions of laminar flow at 2 dynes/cm² was used since it is the rate of laminar flow at postcapillary venules where leukocytes migrate. Spleen cells were added at concentrations below saturation of monolayer cell binding or migration. To initiate spleen cell contact with the endothelial cells in vitro, the spleen cells were allowed to settle in the chamber as monitored by microscopy and then 2 dynes/cm² was applied for the 15 minute laminar flow assay. The co-culture was exposed to laminar flow at 2 dynes/cm² at 37° C. for 3 minutes to examine spleen cell association with the endothelial cells, or for 15 minutes to examine spleen cell migration. After the 3 or 15 minutes at 2 dynes/cm², the cells are washed at 2 dynes/cm² with PBS/0.2 mM CaCl²/0.1 mM MgCl², since cations are required for cell adhesion. The slide was removed from the flow chamber and the cells were fixed with 3% paraformaldehyde for 1 hour. To quantify migrated spleen cells at 15 minutes, phase contrast microscopy was used to count migrated cells that are phase dark. Under laminar flow, spleen cell migration was detected at 15 minutes. The number of spleen cells that were associated but not migrated (phase light cells) at 15 minutes is low since in 15 minutes, the majority of non-migrating cells roll off the monolayer of endothelial cells as determined by microscopy. Therefore, the number of spleen cells associated with the endothelial cells at 3 minutes of laminar flow are those cells that mediated cell-cell contact.

Statistics. Data were analyzed by a one way ANOVA followed by Tukey's multiple comparisons test (SigmaStat, Jandel Scientific, San Ramon, Calif.).

### Example 2

### α- and γ-tocopherol have Opposite Effects on Leukocyte Infiltration and Airway Responsiveness

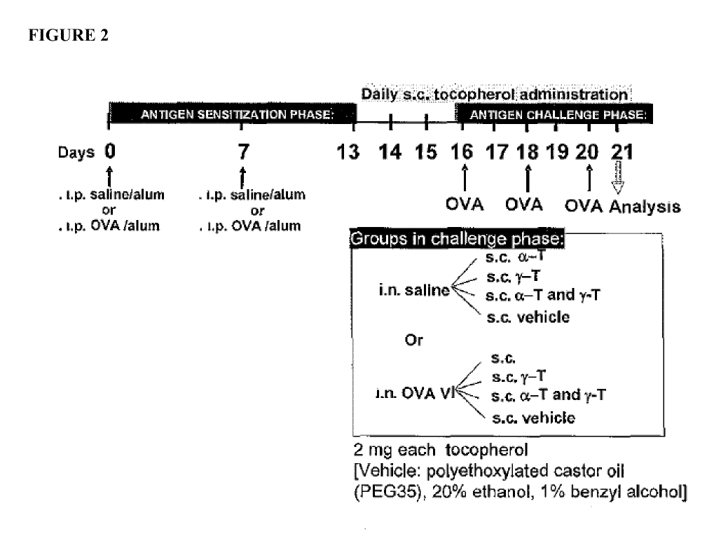

Experiments were conducted during the development of embodiments of the present invention to determine the effect of α- and γ-tocopherol leukocyte infiltration in vivo. Purified natural forms of the tocopherols rather than a mixture of the multiple racemic forms of synthetic tocopherols were used. Tocopherol, rather than water soluble antioxidant vitamins, was choosen since tocopherols are lipid vitamins that are incorporated into membranes, the location of ROS production by NADPH oxidase. Experiments relied upon ascorbic acid is endogenously synthesized by mice, which recycles tocopherols, rather than supplemental ascorbic acid. In the experiments conducted during the development of embodiments of the present invention, daily subcutaneous injections of tocopherols, rather than feeding for weeks with tocopherol-containing diets, was used to delineate whether after antigen sensitization, tocopherols can modulate the antigen challenge phase of experimental asthma. This is especially important since asthmatics responding to antigen are already sensitized. Therefore, during the experimental asthma model, to raise tissue levels of tocopherol between OVA sensitization on day 7 and OVA challenge on day 16 (SEE FIG. 2), a few days of subcutaneous tocopherol administration is adequate. To administer the tocopherols to the mice, tocopherol is placed in the vehicle ethoxylated castor oil. Ethoxylated castor oil is a vehicle used in drug formulations for injections of viscous lipids as it has little side effects. Purified natural d-α-tocopherol or purified natural d-γ-tocopherol was used, which differ by one methyl group (SEE FIG. 1). The purity of these tocopherols was verified by HPLC with electrochemical detection.

Mice were sensitized with intraperitoneal injection of OVA grade V (10 μg)/alum or saline/alum on days 0 and 7 (SEE FIG. 2). On days 13-20, these mice received daily subcutaneous injections (500) of 2 μg natural tocopherol/ethoxylated castor oil (33) or ethoxylated castor oil alone. This daily 2 μg of natural tocopherol is equivalent to the amount of tocopherol that mice consume from diets (4 g diet consumed/day×500 mg a tocopherol/kg diet) that are commonly used to elevate tissue tocopherols in mice. On days 16, 18, and 20 the mice were challenged with intranasal OVA fraction VI (150 μg) in saline or saline alone. On day 21, the mice were analyzed for airway responsiveness and then tissues were collected. Lung responsiveness was determined using methacholine and whole-body plethysmography as previously described. This method of measuring lung function has the advantage of analysis of larger numbers of animals, as compared to the few animals that can be analyzed using invasive measures of lung function. The whole-body plethysmography correlates well with airway function when performed under continuous flow conditions. This measurement under continuous flow is critical as it avoids the complication of airway conditioning that occurs if these measurements are incorrectly made under closed conditions. After airway analysis, mice were sacrificed and tissues collected.

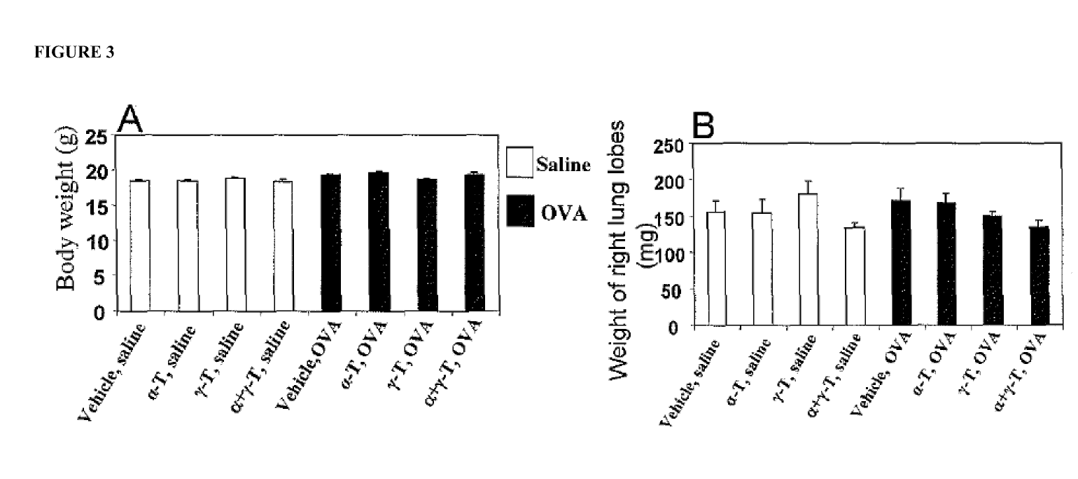

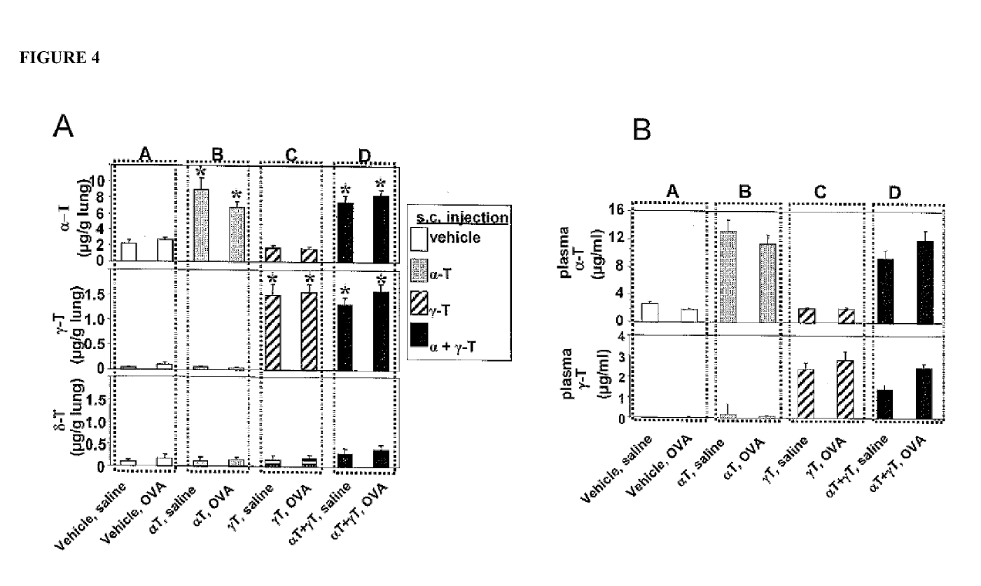

Body weight and tocopherol levels in these mice were examined. It has been reported that dietary α-tocopherol does not affect body weight. Consistent with this, the body weight and lung weights of the mice studied during the development of embodiments of the present invention were unaltered by the α-tocopherol or γ-tocopherol treatments (SEE FIG. 3). Lungs were perfused to remove the blood, homogenized in ethanol and extracted with hexane. Tocopherol concentrations were determined by HPLC with an electrochemical detector. The lung tissue and plasma levels of tocopherols were elevated (SEE FIG. 4). The α-tocopherol-treated mice had elevated levels of lung tissue α-tocopherol. The γ-tocopherol treated mice had elevated levels of lung tissue γ-tocopherol. The level of γ-tocopherol in the lung tissue of γ-tocopherol-treated mice was 10 fold lower than lung tissue α-tocopherol in the α-tocopherol-treated mice. This 10 fold difference in α-tocopherol and γ-tocopherol levels in the lung tissues may be caused preferential transfer of α-tocopherol in the liver by a TTP (20, 24), although the present invention is not limited to any particular mechanism of action and an understanding of the mechanism of action is not necessary to practice the present invention. The α-plus γ-tocopherol-treated mice had the elevated levels of tissue α- and γ-tocopherol. Furthermore, treatment with α- or γ-tocopherol did not alter the lung tissue levels of each other. Administration of tocopherols also had no effect on the low levels of δ-tocopherol in the lung (SEE FIG. 4A).

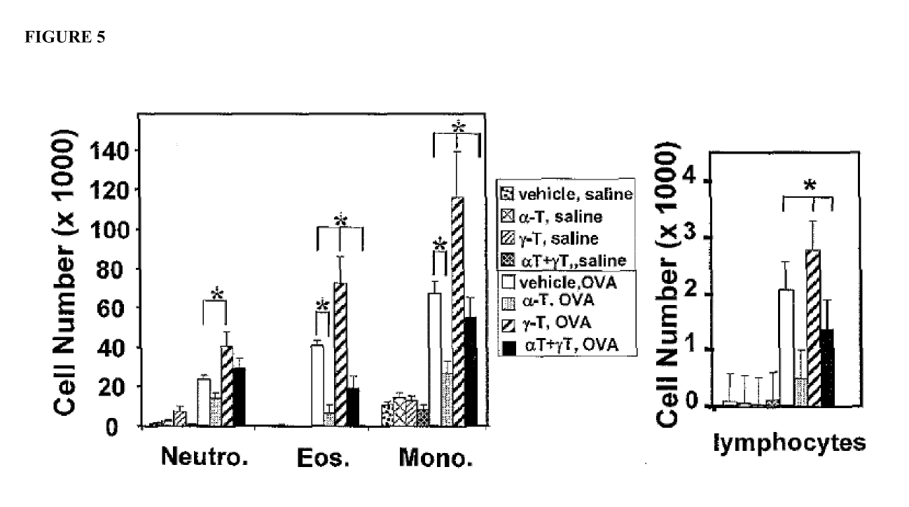

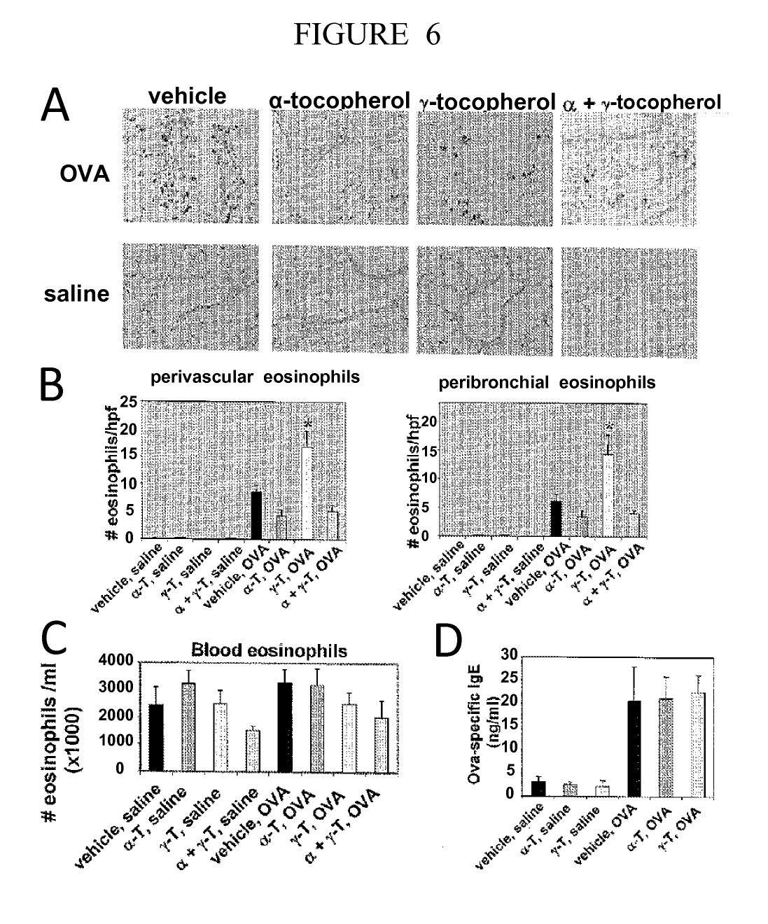

Tocopherol treatments had significant opposing effects on OVA-stimulated inflammation. The infiltration of eosinophils is dependent on VCAM-1 and the infiltration of monocytes, lymphocytes and neutrophils is mediated by ICAM-1 in experimental asthma. α-tocopherol significantly reduced the OVA-stimulated infiltration of eosinophils, lymphocytes and monocytes into the bronchoalveolar lavage (BAL) compared to OVA-challenged mice receiving vehicle only (SEE FIG. 5). OVA-challenged γ-tocopherol-treated mice had significantly increased infiltration of the eosinophils, neutrophils, lymphocytes and monocytes in the BAL compared to OVA-challenged vehicle-treated mice (SEE FIG. 5). For the OVA-challenged mice, γ-tocopherol treatment blocked the benefit of α-tocopherol so that in the α-plus γ-tocopherol-treated mice, there was an intermediate phenotype similar to vehicle-treated mice (SEE FIG. 5). In lung tissue, α-tocopherol reduced eosinophils in the lung tissue and γ-tocopherol elevated lung tissue eosinophils (SEE FIGS. 6 A,B). There were sufficient numbers of eosinphils in the blood available for migration (SEE FIG. 6C). OVA-specific antibodies, which are induced during the OVA sensitization phase of experimental asthma, were not affected by these tocopherols that were administered after the sensitization phase SEE FIG. 6D). α-tocopherol and γ-tocopherol had opposing effects on leukocyte infiltration into the lung in response to OVA.

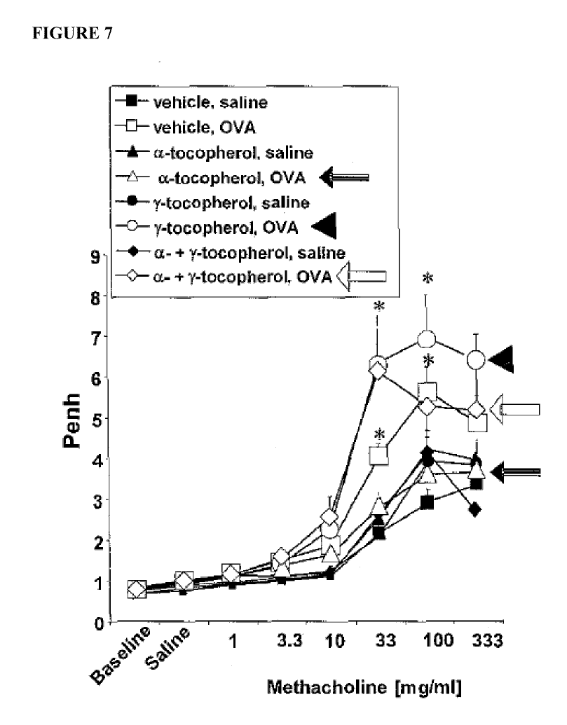

Lung responsiveness in these mice was studied using a dose curve of methacholine inhalation. Vehicle/OVA-treated mice had an increase in airway responsiveness as measured by enhanced pause (Penh) in the BALB/c mice compared to saline-treated control groups (SEE FIG. 7). This increase was completely ablated by administration of d-α-tocopherol (SEE FIG. 7). For experiments performed during development of the embodiments of the present invention, the mice were maintained on standard commercial rodent chow containing basal levels of tocopherols (a-tocopherol (30±6 mg/kg diet), γ-tocopherol (10±1 mg/kg diet) (38) and the mice received subcutaneous injections of the natural forms of >99% pure d-α- or d-γ-tocopherol between the OVA sensitization and OVA-challenge phases (SEE FIG. 2). γ-tocopherol elevated AHR (SEE FIG. 7) and γ-tocopherol antagonized the inhibition by α-tocopherol (SEE FIG. 7). Furthermore, the tocopherol modulation of leukocyte infiltration (SEE FIG. 5) is consistent with the α-tocopherol ablation of AHR and the γ-tocopherol elevation of AHR (SEE FIG. 7).

### Example 3

### Administration of Tocopherol after Sensitization but Before Challenge

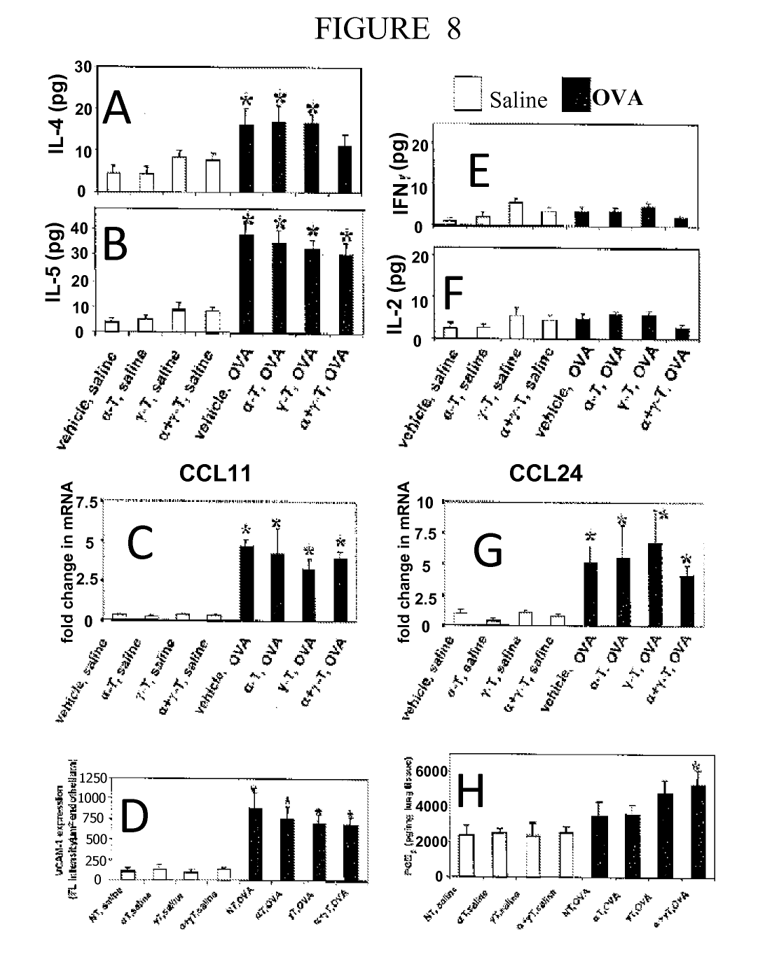

Cytokines produced by resident lung cells (macrophages, mast cells, epithelial cells) and infiltrating leukocytes stimulate endothelial cells to express adhesion molecules and regulate the activation of the leukocytes. Experiments performed during the development of the present invention demonstrated α-tocopherol blocked, whereas γ-tocopherol elevated, infiltration of leukocytes in experimental asthma. Experiments were performed during the development of the present invention to determine whether a selected set of cytokines, chemokines and adhesion molecules determined whether regulate leukocyte infiltration in experimental asthma are altered with the tocopherol treatment of the mice. The cytokines IL-4 and IL-5 were examined since they stimulate eosinophil infiltration. The cytokines IL-2 and 1FNγ were also examined since they downregulate signals for OVA-induced leukocyte infiltration. The adhesion molecule VCAM-1 was examined since it regulates eosinophil and lymphocyte infiltration in the OVA model. The chemokines eotaxin 1 and eotaxin 2 were examined since they stimulate eosinophil chemotaxis into the lung and eosinophilia is a consistent feature of asthma OVA-treated mice expressed the Th2 cytokines IL-4 and IL-5, chemokines eotaxin 1 and eptaxin, and the adhesion molecule VCAM-1 (SEE FIGS. 8A,B,C,D,G). However, the tocopherols did not cause overt changes in these OVA-stimulated mediators (SEE FIGS. 8A,B,C,D,G). The tocopherols did not switch the Th2 cell response to a Th1 response, since, in the tocopherol-treated mice, there was no induction of expression of the Th1 cytokines IFNy and IL-2 (SEE FIGS. 8E,F). Administration of tocopherols after antigen sensitization, but before antigen challenge did not significantly alter the BAL levels of cytokines, the tissue levels of eotaxins, or lung endothelial cell VCAM-1 expression.

To further examine mechanisms for γ-tocopherol elevation of lung inflammation, PGE2 levels in the lung were examined. It has been reported that in vitro, tocopherols inhibit COX2 and therefore PGE₂ synthesis. In experiments performed during the development of the present invention PGE₂ was examined, the ethanol fraction from the ethanol/hexane extracts was collected, dried under nitrogen and resuspended in the High Sensitivity PGE₂ ELISA kit buffer (Cambridge Siosciences). The OVA-challenged lungs had a trend of an increase in PGE₂ compared to the saline controls but, there was no reduction in PGE₂ in the γ-tocopherol-treated groups (SEE FIG. 8H). Therefore, γ-tocopherol's reported in vitro non-antioxidant function of COX2 inhibition does not play a significant role in this model of experimental asthma.

### Example 4

### α- and γ-tocopherol have Opposite Effects on Leukocyte Migration In Vitro

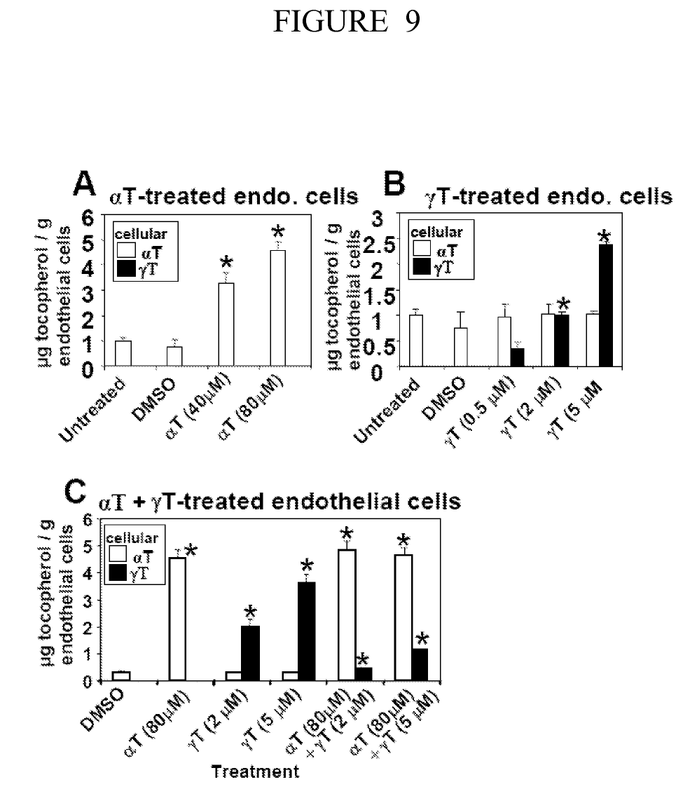

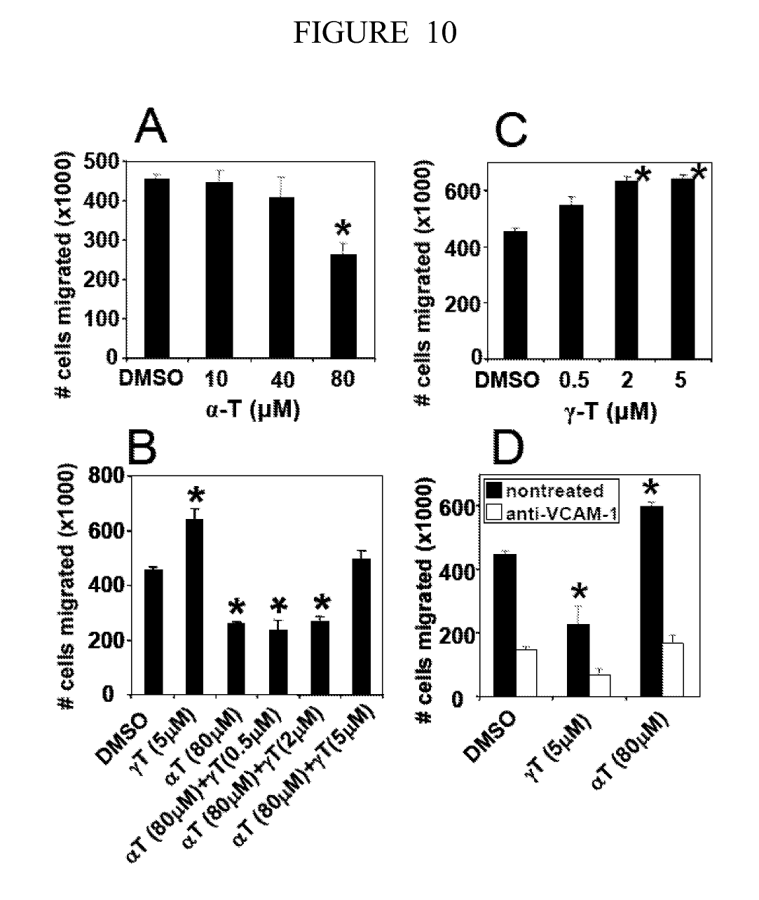

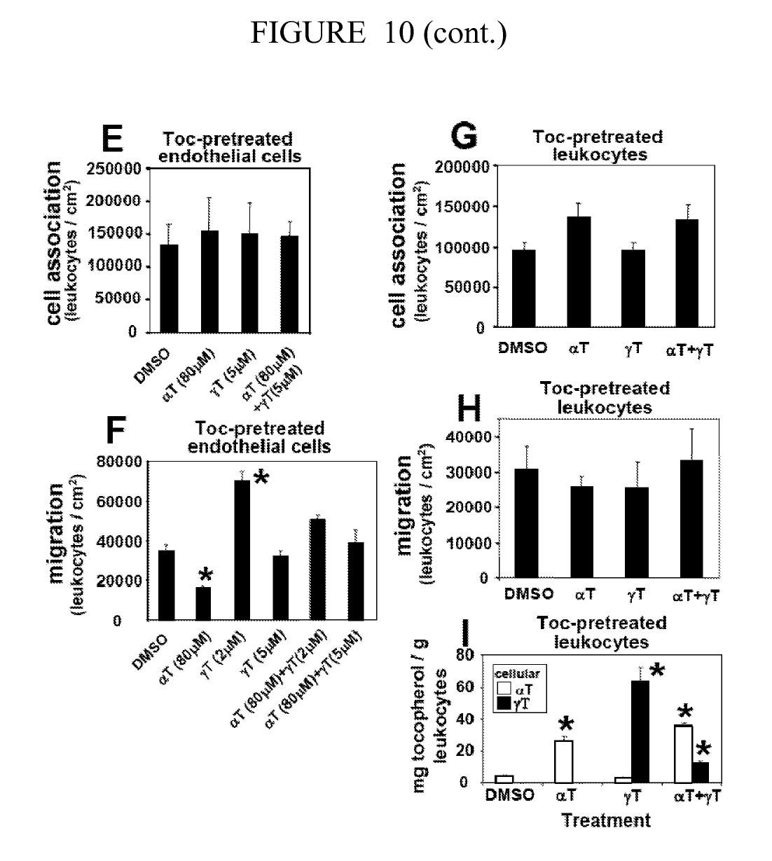

Experiments were conducted during the development of the present invention to determine whether α-tocopherol or γ-tocopherol modulate the transendothelial migration event. An in vitro endothelial cell model of adhesion molecule-dependent and chemokine-dependent leukocyte migration was used to address the effects of forms of purified tocopherols on leukocyte transendothelial migration. Experiments conducted during the development of the present invention determined that in vitro, 5 μM γ-tocopherol in the culture medium for 18 hours raised the endothelial cell concentration of γ-tocopherol (SEE FIG. 9B) to that in the lung tissue of tocopherol-treated mice (SEE FIG. 4). In addition, in vitro 80 μM α-tocopherol in the culture medium for 18 hours raised the endothelial cell concentration of α-tocopherol five fold (SEE FIG. 9A) which is similar to the five-fold increase in α-tocopherol in lungs of α-tocopherol-treated mice (SEE FIG. 4). The absolute value for endothelial cell concentration of α-tocopherol in vitro did not quite reach that for the lungs tissue but there was a five-fold increase in vitro and in vivo. Nevertheless, at this five-fold increase in α-tocopherol level, α-tocopherol inhibited lymphocyte migration in a transwell migration assay (SEE FIG. 10) whereas d-γ-tocopherol significantly increases lymphocyte migration 24 hours after addition of the spleen cells (SEE FIG. 10). The vehicle DMSO (0.02%) had no effect on migration. Treatment with 5 μM γ-tocopherol ablated the α-tocopherol inhibition of spleen cell migration (SEE FIG. 10). These tocopherols did not alter the VCAM-1 dependence of the migration (SEE FIG. 10) as demonstrated by treatment with blocking anti-VCAM-1 antibodies.

### Example 5

### The Effect of Tocopherols on Endothelial Cells

Experiments were performed during the development of the present invention to determine whether the tocopherols had a direct effect on the endothelial cells and/or on the spleen cells. In this transendothelial migration assay used, the migrated spleen cells are >95% lymphocytes and the B cells and T cells migrate in response to MCP-1. The endothelial cells were pretreated with tocopherols, washed five times and then used in the transwell migration assay. Leukocytes were pretreated with a- or v-tocopherol and washed, there was no effect on migration (SEE FIG. 10). Pretreatment of the endothelial cells with γ-tocopherol elevated lymphocyte migration in the transwell assay, indicating that γ-tocopherol has a direct effect on the endothelial cell function during leukocyte migration (SEE FIG. 10).

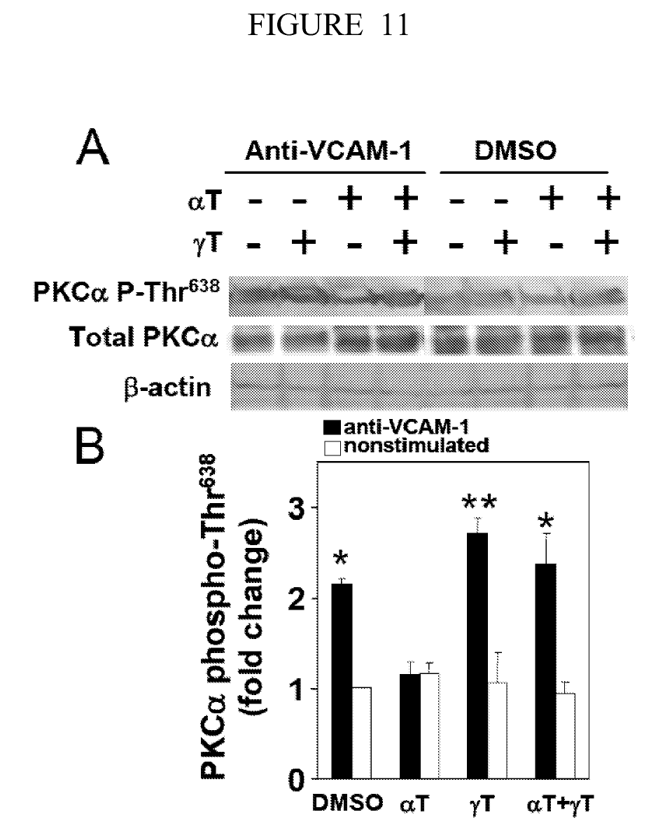

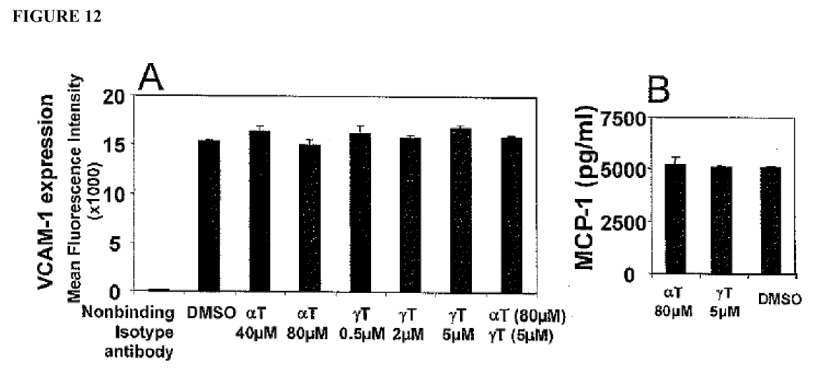

Experiments were performed during the development of the present invention to determine whether α-tocopherol and γ-tocopherol have a direct effect on endothelial cells using a transendothelial migration assay with physiological laminar flow. The endothelial cells were pretreated overnight with d-α-tocopherol, washed, and then lymphocyte transendothelial migration was examined for 15 minutes under laminar flow at 2 dynes/cm² using a parallel plate flow chamber (SEE FIG. 11), the rate of blood flow at post-capillary venules where leukocytes migrate. The d-α-tocopherol pretreatment of endothelial cells inhibited VCAM-1-dependent lymphocyte migration, demonstrating a direct effect of d-a-tocopherol on endothelial cells (SEE FIG. 11). The vehicle control, DMSO (0.02%), did not affect migration. The tocopherols and vehicle controls had no effect on cell viability as determined by tiypan blue exclusion. There was no effect of tocopherols on endothelial cell expression of VCAM-1 as determined by immunolabeling of the endotheial cells and flow cytometry (SEE FIG. 12A). Thus, in vitro (SEE FIG. 12A) and in vivo (SEE FIG. 8D), the tocopherols did not alter VCAM-1 expression. There was also no effect of the tocopherols on lymphocyte adhesion to the endothelial cells.

Experiments were performed during the development of the present invention to examine the endothelial cell-derived expression of the chemokine MCP-1 in the tocopherol-treated transwell samples during leukocyte migration. There was no effect of tocopherols on the MCP-1 expression as determined by the CBA Inflammation kit (BD Biosciences) and flow cytometry (SEE FIG. 12B). The tocopherols had a direct effect on endothelial cell function for the promotion of leukocyte migration without altering endothelial cell adhesion molecule expression or chemokine expression. There was no effect of tocopherols on the lymphocytes during transendothelial migration.

### REFERENCES

The following references are herein incorporated by reference in their entireties.
Abdala-Valencia et al. 2007. Am. J. Physiol. 292: L1111-L1125.
Abdala-Valencia & Cook-Mills. 2006. J. Immunol. 177: 6379-6387.
Deem et al. J. Immunol. 178: 3865-3873.
Deem & Cook-Mills. 2004. Blood 104: 2385-2393.
Kalayci et al. 2000. Turk. J. Peds. 42: 17-21.
Kelly et al. 1999. Lancet 354: 482-483.
Shvedova et al. 1995. Toxicol. Appl. Pharmacol. 132: 72-81.
Schunemann et al. 2001. Am. J. Respir. Crit. Care Med. 163: 1246-1255.
Tabak et al. 1999. Thorax 54: 1021-1026.
Smit et al. 1999. Proc. Nutr. Soc. 58: 309-319.
Weiss. 1997. Ciba Found. Symp. 206:244-257.
Troisi et al. 1995. Am. J. Respir. Crit. Care Med. 151: 1401-1408.
Dow et al. 1996. Am. J. Respir. Crit. Care Med. 154: 1401-1404.
Siekmeier et al. 2007. J. Cardiovasc. Pharmacol. Ther. 12: 265-282.
Dietrich et al. 2006. J. Am. Coll. Nutr. 25: 292-299.
Wagner et al. Ann. Nutr. Metab. 48: 169-188.
Wolf. 2006. Nutr. Rev. 64: 295-299.
Desrumaux et al. 1999. FASEB J. 13: 883-892.
Matheny et al. 2000 J. Immunol. 164: 6550-6559.
Mustacich et al. 2006. Free Radical Biol. Med. 41: 1069-1078.
Hayek et al. 1997. J. Infect. Dis. 176: 273-276.
Meydani et al. 1992. J. Nutr. 122:1627-1633.
Bryce et al. 2003. J. Allergy Clin. Immunol. 112: 149-158.
Bryce et al. 2006. J. Clin. Invest. 116: 1624-1632.
Lomask. 2006. Exp. Toxicol. Pathol. 57 (Suppl. 2): 13-20.
Finkelman. 2008. J. Allergy Clin. Immunol. 121: 334-335.
Tudor et al. 2001. Cytokine 15: 196-211.
Qureshi et al. 2003. J. Immunol. 171:4700-4707.
Yamamoto et al. 1998. J. Immunol. 161: 971-977.
May & Ager. 1992. Eur. J. Immunol. 22: 219-226.
Tsao & Young. 1990. In Vivo 4: 167-169.
Weber et al. 1997. Free Radical Biol. Med. 22: 761-769.
Meydani et al. 1987. Lipids 22: 345-350.
Sandig et al. 2007. J. Leukocyte Biol. 81: 372-382.
Wu et al. 1999. Atherosclerosis 147: 297-307.
O′Leary et al. 2004. Mutat. Res. 551: 245-254.
Sakamoto et al. 1991. Biochim. Biophys. Acta 1074: 251-255.
Wu et al. 2000. Free Radical Biol. Med. 28: 643-651.
Egger et al 2003. Biochem. J. 370: 459-467.
Mine et al. 2002. Atherosclerosis 160: 281-288.
Meydani et al. 1997. J. Am. Med. Assoc. 277: 1380-1386.
Stahelin et al. 1989. Ann. NY Acad. Sci. 570: 391-399.
Martin et al. 1996. Free Radical Biol. Med. 20: 99-105.
Tran & Chan. 1992. Lipids 27: 38-41.
Azzi & Stocker. 2000. Prog. Lipid Res. 39: 231-255.
Keshavan et al. J. Immunol. 174: 3709-3718.
Pero et al. 2007. Proc. Natl. Acad. Sci. USA 104: 4371-4376.
Jiang et al. 2001. Am. J. Clin. Nutr. 74: 714-722.
Jaudszus et al 2008. J. Nutr. 138: 1336-1342.
Knapp et al. 2006. J. Immunol. 176: 3189-3195.
McKeever et al. 2008. Thorax 63: 208-214.
Almqvist et al. 2007. J. Allergy Clin. Immunol. 119: 1438-1444.
Mickleborough et al. 2004. J. Altern. Complement Med. 10:1067-1075.
Friebele. 1996. Health Perspect. 104: 22-25.
Vollmer et al. 1998. Am. J. Respir. Crit. Care Med. 157: 1079-1084.
Van Schayck & Smit. 2005. Eur. Respir. J. 26: 647-650.
Ait-Khaled et al. 2001. Bull World Health Organ 79: 971-979.
Bousquet et al. 2005. Bull World Health Organ 83: 548-554.
Jordan et al. 2004. Am. J. Epidemiol. 159:968-977.
Brand et al. 2001. Ann. Rheum. Dis. 60: 946-949.
Munteanu et al. 2004. J. Cell Mol. Med. 8: 59-76.
Munteanu & Zingg. 2007. Mol. Aspects Med. 28: 538-590.
Meydani. 2004. Ann. NY Acad. Sci. 1031: 271-279.
Dutta & Dutta. 2003. J. Am. Coll. Nutr. 22: 258-268.
Suchankova et al. 2006. Respirology 11: 414-421.
Okamoto et al. 2006. Int. Arch. Allergy Immunol. 141: 172-180.
Lee et al. 2006. Carcinogenesis 27: 2308-2315.
Wagner et al. 2008. Clin. Exp. Allergy 38: 501-511.
Wagner et al. 2007. Free Radic. Biol. Med. 43: 1176-1188.
Lim et al. 2008. Free Radic. Res. 42: 387-396.
Jung & Sung. 2004. Biofactors 21: 113-117.
Konger. 2006. J. Invest. Dermatol. 126: 1447-1449.
Jiang & Ames. 2003. FASEB J. 17:816-822.
Wu et al. 2005. J. Nutr. 135:1847-1853.
Sakamoto et al. 1996. Biochim. Biophys. Acta. 1304: 139-144.
Grammas et al. 2004. Biochem. Biophys. Res. Commun. 319: 1047-1052.
Jiang. 2000. Proc. Natl. Acad. Sci. USA 97:11494-11499.
Jiang et al. 2008. J. Nutr. Biochem. [Nov. 6, 2008 Epub ahead of print.]
Zapolska-Downar et al. 2000. Biochem. Biophys. Res. Commun. 274: 609-615.
Erl et al. 1997. Am. J. Physiol. 273: H634-H640.
Pietersma et al. 1998. Free Radic. Res. 28: 137-150.
Willam et al. 1999. Am. J. Physiol. 276: H2044-H2052.
Jialal et al. 2001. J. Nutr. 131: 389S-394S.
Vega-Lopez et al. 2004. Metabolism 53: 236-240.
Navab et al. 1988. J. Clin. Invest. 82: 1853-1863.

1. A method of treating inflammation in a subject comprising:
a) providing a tocopherol composition wherein said tocopherol composition comprises greater than 90% α-tocopherol and less than 10% γ-tocopherol; and
b) administering said tocopherol composition to said subject under conditions such that inflammation in said subject is reduced.

2. The method of claim 1, wherein said tocopherol composition comprises greater than 95% α-tocopherol and less than 5% γ-tocopherol.

3. The method of claim 1, wherein said tocopherol composition comprises greater than 99% α-tocopherol and less than 1% γ-tocopherol.

4. The method of claim 1, wherein said tocopherol composition comprises greater than 99.9% α-tocopherol and less than 0.1% γ-tocopherol.

5. The method of claim 1, wherein said tocopherol composition comprises a delivery vehicle, wherein said tocopherol delivery vehicle may contain additional components configured to deliver said tocopherol composition to said subject.

6. The method of claim 5, wherein said delivery vehicle comprises an oral delivery vehicle.

7. The method of claim 5, wherein said delivery vehicle comprises a food supplement.

8. The method of claim 5, wherein said delivery vehicle comprises a food product.

9. The method of claim 5, wherein said tocopherol delivery vehicle comprises a dietary supplement.

10. The method of claim 5, wherein said tocopherol delivery vehicle comprises a drug vehicle.

11. The method of claim 5, wherein said delivery vehicle comprises a pharmaceutical compositon.

12. The method of claim 1, wherein said inflammation results from asthma.

13. The method of claim 1, wherein said inflammation results from atherosclerosis.

14. A composition comprising a food product, wherein said composition is supplemented by the addition of the α-tocopherol isoform of vitamin E relative to the γ-tocopherol isoform.

15. The composition of claim 14, wherein said vitamin E is substantially free of γ-tocopherol.

16. The composition of claim 14, wherein said composition comprises a food product.

17. The composition of claim 14, wherein said composition comprises a pharmaceutical.

18. The composition of claim 14, wherein said composition comprises a dietary supplement.
